(19) United States
(12) Patent Application Publication
(10) Pub. No.: US 2010/0318041 A1
(43) Pub. Date: Dec. 16, 2010

(54) METHOD AND APPARATUS FOR SINGLE-USE CREAM OR LIQUID APPLICATOR

(75) Inventor: Carman Cook, Canton, GA (US)

Correspondence Address: R. Scott Griffin, Esq.;R. Scott Griffin Law, LLC, 2964 Peachtree Road, NW, Suite 720, Atlanta, GA, 30305, US

(73) Assignee: C SMART PRODUCTS, LLC, Atlanta, GA (US)

(21) Appl. No.: 12/797,300

(22) Filed: Jun. 9, 2010

Related U.S. Application Data
(60) Provisional application No. 61/268,368, filed on Jun. 11, 2009.

(51) Int. Cl. A61M 35/00

(52) U.S. Cl. 604/290; 604/310

(57) ABSTRACT

A method and apparatus for applying a cream, lotion, medicament, ointment, or other liquid from a disposable, single-use applicator that made be used with one hand. The assembly includes a flexible, low-rigidity container adapted to carry the agent therein. The assembly is formed with a container portion and an applicator tip, with the agent held inside by a perforated seal which is broken with a squeeze.

### RELATED APPLICATIONS AND CLAIM OF PRIORITY

This application is related and claims priority to U.S. provisional patent application No. 61/268,368, filed Jun. 11, 2009.

### BACKGROUND OF THE INVENTION

1. Field of the Technology

This invention relates to the field of one-time use applicators for ointments, creams, treatments and any other liquid, gel, paint, or make-up.

2. Background

A ubiquitous and near-constant condition for any baby or child still in diapers is diaper rash. Many individuals are afflicted with skin disorders such as eczema, psoriasis, skin allergies, and many forms of rash-inducing conditions. There is a wide range of treatment agents for these conditions, such as steroid lotions and ointments or salves containing Vitamin E, aloe, and cortisone, among many others. These agents are most frequently packaged in tubes with caps, like a tube of toothpaste. The tube containers themselves may be constructed of a variety of materials from plastic to metals and with varying rigidity. There are multiple problems with such packaging. First, caps frequently become dirty and clogged with unused or excess agent. Not only is this wasteful, but it also makes the dispenser unsanitary to use. Second, many such dispenser tubes are large and/or cumbersome when attempting to use them one-handed, in tight quarters, or quickly and on the go. In many cases, one may find him or herself wishing for an extra hand.

There are additional problems with the size of traditional packaging. First the tubes are often large and add to the bulk that individuals must carry with them on a daily basis. With specific regard for parents of diaper-aged children, these tubes are added weight and volume to already-full diaper bags. This problem is not limited to diaper-rash creams and overburdened parents, but modern life has led to more and more individuals carrying many medications, ointments, lotions, medicaments, and any number of other liquid items with them on a daily basis. Therefore, many agents in traditional packaging create extra weight and bulk in already-crowded bags. The current state air travel also limits the amount of luggage passengers carry. Fees for checking luggage result in more carry-on baggage, and lotions, liquids, and creams are required to be limited in volume and in the amount one person can carry. In addition to treatments and agents for diaper rash, other ointments, and make-up, which are used topically, items such as shoe polish, paint samples, and any number of other liquids and/or creams may come in cumbersome, bulky, or wasteful packaging. Many such items, an exhaustive list of which would be too lengthy to include, here, would benefit from a small, compact, single-use packaging alternative.

There exists a need, therefore, for small, compact, and clean dispenser that may be used with one hand for lotions, ointments, creams, polishes, make-up, Armor-All®, and another substance for which such a dispenser would be convenient. There exists a need for such a package that is less cumbersome than traditional packaging and sized for the necessary dosage or application amount sufficient for a one-time use, depending on the substance or agent. Such a package would avoid waste and unsanitary caps. Such a package would have a built-in applicator, further avoiding a mess and the need for articles to apply the agent from the dispenser or to clean up following use. After the use, the dispenser and applicator may then simply be disposed of in a trash receptacle.

In accordance with an embodiment of the invention, a preferred embodiment of the invention is comprised of a one-time use dispenser for applying an agent comprised of a low-rigidity agent containment chamber; a pathway; a neck seal disposed within said pathway and adjacent to said low-rigidity containment chamber; and an applicator fixed to said pathway.

A further embodiment of the invention includes a one-time use dispenser, wherein said neck seal is a perforated plastic film.

A further embodiment of the invention includes a one-time use dispenser, further comprising a closure cap which engagably seals to said dispenser body.

A further embodiment of the invention includes a one-time use dispenser, wherein said applicator is further comprised of an opening disposed on a surface through which said pathway may be inserted and attached to said applicator.

A further embodiment of the invention includes a one-time use dispenser wherein said applicator is further comprised of slits disposed on a surface.

A further embodiment of the invention includes a method for applying an agent comprising providing a on-time use dispenser comprising: a low-rigidity agent containment chamber; a pathway; a neck seal disposed within said pathway and adjacent to said low-rigidity containment chamber; and an applicator fixed to said pathway.

A further embodiment of the invention includes a method for applying an agent, further comprising providing neck seal is a perforated plastic film.

A further embodiment of the invention includes a method for applying an agent, further comprising providing a closure cap which engagably seals to said dispenser body.

A further embodiment of the invention includes a method for applying an agent further comprising providing an opening disposed on a surface of an applicator through which said pathway may be inserted and attached to said applicator.

A further embodiment of the invention includes a method for applying an agent further providing an applicator further comprised of slits disposed on a surface.

A further embodiment of the invention includes a method for applying an agent further comprising squeezing said neck seal.

A further embodiment of the invention includes a method for applying an agent further comprising squeezing said containment chamber.

### BRIEF DESCRIPTION OF THE DRAWINGS

FIG. 1 is a front, cut-away view of an embodiment of the invention having an applicator with a closure cap assembly.

FIG. 2 is a side view of a prior art dispenser that is improved upon in an embodiment of the invention showing the agent pathway and tip to which an applicator attaches.

FIG. 3 is a prior art, top, cross-sectional view of an agent pathway and tip on which an applicator attaches, which is improved upon in an embodiment of the invention.

FIG. 4 is a side view of an embodiment of the invention showing an agent pathway and tip on which an applicator attaches with a foam applicator being attached to it.

FIG. 5 is a close-up view of an embodiment of the invention showing a foam applicator, with a specific view of the bottom of the applicator where the agent pathway and tip is inserted for attachment.

FIG. 6 is an alternative embodiment of the dispenser of the invention showing an alternative dispenser neck and agent pathway and with the dispenser being open at its base so it may be filled.

FIG. 7 is an alternative embodiment of the dispenser of the invention showing an alternative dispenser neck and agent pathway and with the dispenser being sealed at its base.

FIG. 8 is a top view of the alternative embodiment of the dispenser of the invention showing a perforated seal in the neck of the dispenser, which contains the liquid agent.

FIG. 9 shows an embodiment of the invention in use to treat diaper rash.

### DETAILED DESCRIPTION OF EXEMPLARY EMBODIMENTS

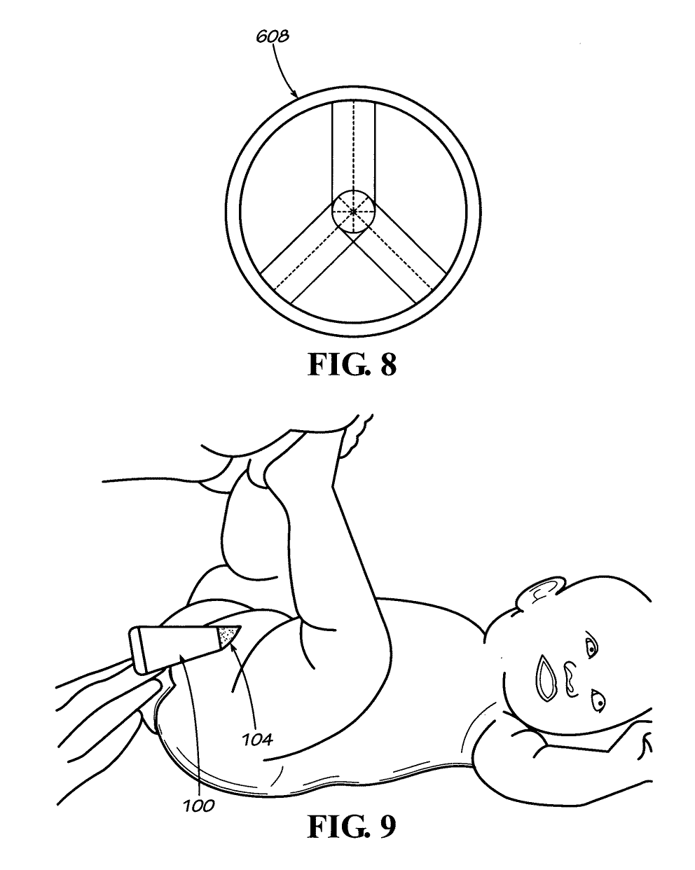

Turning now to the drawings, where like numbers represent like elements, FIG. 1 is a front perspective, cut-away view of an embodiment of the invention 100 having an applicator 104 and a closure cap assembly 106. For descriptive purposes, the dispenser tube will be made of a low-rigidity material that is easily compressed by the force of a person's fingers. Such a dispenser may be manufactured and produced by the James Alexander Corporation of Blairstown, N.J., which is the owner of prior art U.S. Pat. No. 6,796,737. In its final configuration, the dispenser will consist substantially of a tube, such as for toothpaste or other creams or ointments, with an applicator attached, as shown in FIG. 9, below. Such a tube may also be manufactured by USA JDK, Inc., headquartered in Irving, Tex. It will be understood by one of ordinary skill in the art that any such similar dispenser tube may be used and disclosure is for explanatory purposes, only. Further for descriptive and explanatory purposes only, and not for limitation, the embodiment of the invention described herein contains cream, ointment or other topical agent for the treatment of a skin condition such as diaper rash. The dispenser 100 of the embodiment of the invention contains the agent 102 up to the dispenser neck.

FIG. 1 also shows the foam applicator 104 attached to the packaging tube. The foam applicator is porous, latex-free and made with a Vitamin E, polyurethane hemophilic. The foam may also be manufactured by USA JDK, Inc., and cut into pieces of varying sizes and shapes as necessary and adaptable for the particular agent that it is used to apply. It will be understood by one of ordinary skill in the art that the consistency, thickness, and chemical composition of the foam applicator may change depending on the thickness, viscosity, and texture type of substance being packaged. The foam applicator described herein is for purposes of explanation and description, only, and is not intended as a limitation.

FIG. 1 also shows a closure cap 106, which fits over the applicator and prevents it from leaking Certain embodiments may not use a cap. Rather, one of ordinary skill in the art will understand that the applicator may be shrink-wrapped to prevent leaking, and as discussed below, the dispenser construction also prevents spillage and leaking Also shown is the agent pathway opening 108 in the neck of the dispenser, which holds the agent in the dispenser tube until ruptured. One type of construction by which the agent is held within the dispenser is described in prior art U.S. Pat. No. 6,976,737 to Ashe. Another type of seal at the neck of the dispenser 100 that holds in the agent is shown in FIG. 8, below.

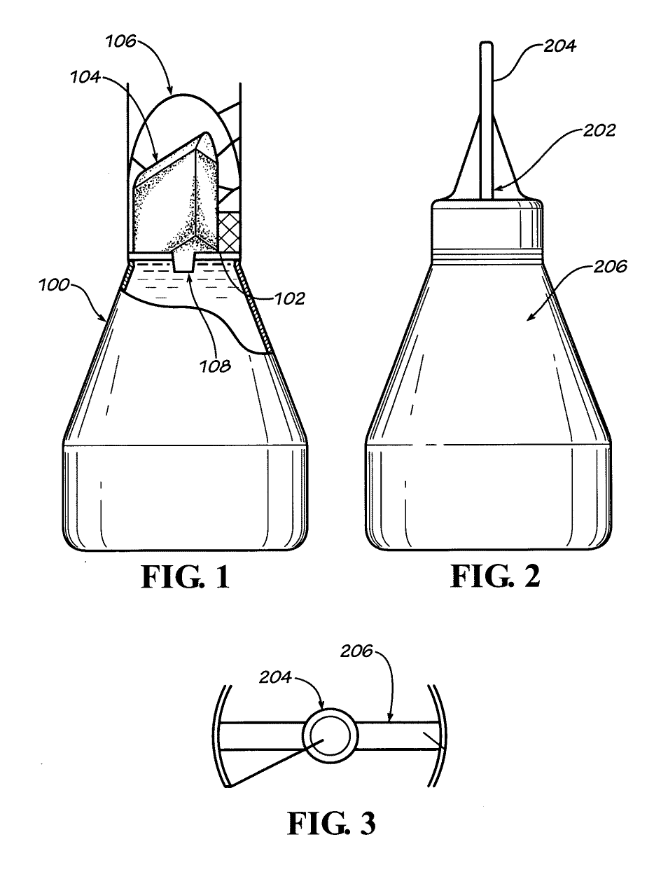

Turning to FIG. 2, the dispenser 100 of an embodiment of the invention is shown without the applicator 104, revealing the pathway 202 through which the agent moves to the applicator 104 when the dispenser is squeezed. No specific equipment is needed to operate the dispenser 100. To use the dispenser 100, the user squeezes the neck of the dispenser 100 just below the applicator, which ruptures the sealed opening 108 at the neck of the dispenser 100. When the user squeezes the dispenser 100, the agent moves along the pathway 202 and to the tip 204, where the applicator 104 is attached to receive the agent. FIG. 3 shows a top view of the pathway tip 204 and side supports 206, on which an applicator 104 may lock when it is placed on this embodiment of the dispenser 100.

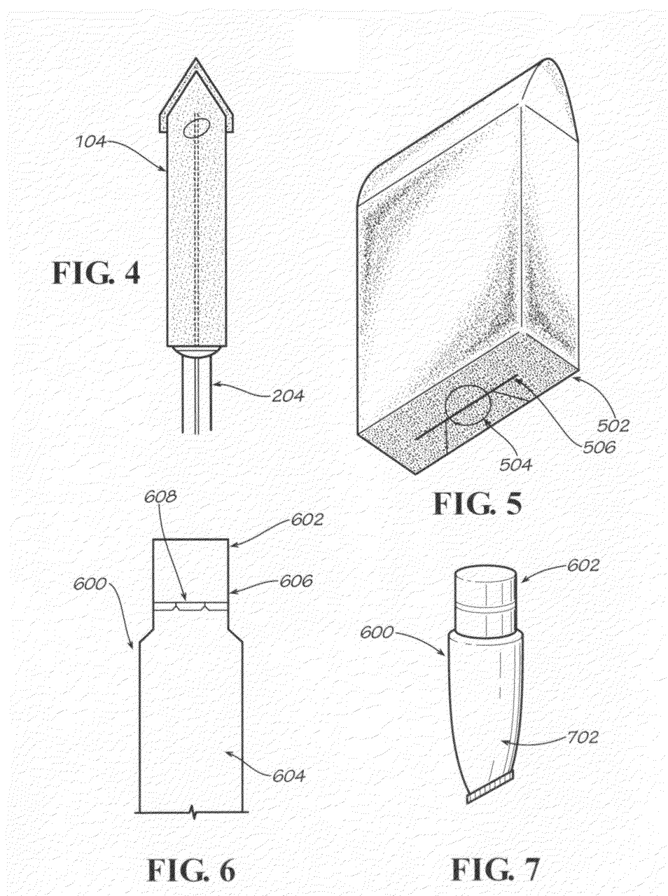

FIG. 4 shows the applicator 104 being placed on the pathway 202 and tip 204 of an embodiment of the invention. FIG. 5 is a close-up of an applicator 104 of this embodiment of the invention. The bottom 502 of the applicator 104 has an opening 504 through which the pathway 204 is placed. In line with the opening 504 are slits 506 that lock on to the side supports 206 for this embodiment. The opening 504 extends the full length of the applicator 104. The applicator 104 is sized such that the pathway 204 substantially fills the length of the applicator through opening 504, which stretches through the other end of the applicator 104. The pathway 204 tip will be covered by the applicator 104, and the agent will move out onto the applicator 104 with the user squeezes the dispenser 100. The agent may then be applied.

FIG. 6 is an alternative embodiment of the dispenser 600 of the invention without the applicator 104 attached showing an alternative dispenser pathway 602 and with the dispenser 600 being open at its base 604 so it may be filled with the agent. The foam applicator 104 is slid on via its opening 504 and attached to the dispenser neck 602 in this embodiment. The agent is filled to the neck 606 of the dispenser. Within the neck 606 is a perforated seal 608. A user operates the dispenser 600 by compressing the neck 606, which breaks the perforated seal 608. When the dispenser 600 is squeezed, the agent moves through the pathway 602 and onto the applicator 104, as it does with the prior-described embodiments.

FIG. 7 is an alternative embodiment of the invention of the dispenser 600 without the applicator attached and showing an alternative agent pathway 602 and with the dispenser being sealed at its base 502. FIG. 8 is a top view of the perforated seal 608 holding the agent in the dispenser 600 until time for use.

FIG. 9 shows an embodiment of the invention in use on a baby. The user squeezes the dispenser 100 such that the cream fills the applicator 104 so that it may be then used on the diaper rash or other skin condition or ailment.

An exemplary embodiment of the present invention addresses the multiple needs for providing a one-time use applicator and dispenser for creams, lotions, ointments, other remedies and medicaments, and any other liquid. The exemplary embodiment of the invention is sanitary, small, light-weight, and easy to pack and carry. The exemplary embodiment may be used with one hand. It will be understood by one of ordinary skill in the art that the above description is for example, only, and not meant as a limitation, and any dispenser and applicator of the types described above may be used and are included in this disclosure.

What is claimed is:

1. A one-time use dispenser for applying an agent comprised of:
a low-rigidity agent containment chamber;
a pathway;
a neck seal disposed within said pathway and adjacent to said low-rigidity containment chamber; and
an applicator fixed to said pathway.

2. The one-time use dispenser of claim 1, wherein said neck seal is a perforated plastic film.

3. The one-time use dispenser of claim 1, further comprising a closure cap which engagably seals to said dispenser body.

4. The one-time use dispenser of claim 1, wherein said applicator is further comprised of an opening disposed on a surface through which said pathway may be inserted and attached to said applicator.

5. The one-time use dispenser of claim 1, wherein said applicator is further comprised of slits disposed on a surface.

6. A method for applying an agent comprising providing a one-time use dispenser comprising:
a low-rigidity agent containment chamber;
a pathway;
a neck seal disposed within said pathway and adjacent to said low-rigidity c containment chamber; and
an applicator fixed to said pathway.

7. The method of claim 6 for applying a treatment agent, wherein said said neck seal is a perforated plastic film.

8. The method of claim 6 for applying a treatment agent, further comprising a closure cap which engagably seals to said dispenser body.

9. The method of claim 6 for applying a treatment agent, wherein said foam applicator is further comprised of an opening disposed on a surface through which said pathway may be inserted and attached to said applicator.

10. The method of claim 6 for applying a treatment agent, wherein said foam applicator is further comprised of slits disposed on a surface.

11. The method of claim 6 further comprising squeezing said neck seal.

12. The method of claim 11 further comprising squeezing said containment chamber.
